(19) United States
(12) Patent Application Publication
(10) Pub. No.: US 2011/0177010 A1
(43) Pub. Date: Jul. 21, 2011

(54) Vitamin preparation

(75) Inventor: Tanja Lautenschläger, Klausdorf/Schwentine (DE)

(73) Assignee: Nutri Team GmbH

(21) Appl. No.: 12/929,156

(22) Filed: Jan. 4, 2011

Related U.S. Application Data
(62) Division of application No. 12/213,295, filed on Jun. 17, 2008, now abandoned.

(30) Foreign Application Priority Data
Nov. 27, 2007 (DE) 20 2007 016 544.7
Nov. 27, 2007 (EP) 07 022 973.7

(51) Int. Cl. A61K 9/48; A61K 33/04; A61K 9/20; A61K 33/30; A61P 3/02

(52) U.S. Cl. 424/10.3; 424/702; 424/464; 424/451; 424/10.1; 424/643

(57) ABSTRACT

Two-phase preparation for temporally gradual administration comprising
a) a phase (A) comprising water soluble vitamins and/or the corresponding provitamins, wherein phase (A) does not contain liposoluble vitamins or provitamins corresponding to liposoluble vitamins,
b) a phase (B) comprising vitamins and/or the corresponding provitamins, wherein the quantity of liposoluble vitamin compounds is larger than the quantity of water soluble vitamin compounds.

The present invention relates to a new vitamin preparation as well as the use thereof.

It is commonly known that nutrients like carbohydrates and nutritional fats mainly serve to cover the energy requirement. Proteins are a fundamental nutrient for body cells and the body's own active agents such as enzymes and certain hormones. Vitamins, minerals, and trace elements are increasingly gaining in importance in health and productivity.

Vitamins, minerals, and trace elements are vital substances in controlling the metabolism and protective nutrients for health. Because of today's living conditions, there is a great risk of an unbalanced diet as well as a preference for food which is rich in calories and has a low density of nutrients, which can lead at times to a poor supply of vitamins, minerals, and trace elements in the human body. Additionally, problems of environmental impact, increased consumption of alcohol and smoke as well as stress in daily life are present nowadays, leading to an increased need for vitamins, minerals, and trace elements.

Vitamins are vital nutritional components which have to be supplied more or less obligatorily and as required for the normal functions of the human body because they are only accessible by an external source or under the control of environmental factors such as the intestinal flora. Their specific biocatalytic effect is based on the substitution of the active groups of enzymes, which are subject to metabolism. It is known in the scientific field that vitamins in the B group act as coenzymes in intermediate metabolism and vitamins C, E, and betacarotin act, in particular, as antioxidants. Malnutrition as a result of insufficient supply or resorption, disruption of the intestinal flora or metabolism, anti-vitamin effect or mutual consumption leads to vitamin deficiencies.

Numerous vitamin preparations are available on the market. These vitamin preparations contain a plurality of different vitamins and are considered to enhance and support performance and regeneration. However, the preparations provided are not balanced in their composition, i.e. individual components of the preparations act competitively instead of supportively, avoiding a positive effect of the individual pharmaceutical active elements.

Therefore, it is the object of the present invention to provide a pharmaceutical preparation that is particularly well suited as a nutritional supplement, which avoids or reduces the interference of single components and thus enhances administration and absorption of constituent nutrients.

The finding of the present invention is to provide a preparation that comprises two separate phases. These phases contain different vitamins and can be administered temporally gradually to exclude negative interactions between individual vitamins. Thus, the present invention is directed, in one aspect, to a two-phase preparation for temporally gradual administration comprising
(a) a phase (A) comprising water soluble vitamins and/or the corresponding provitamins, wherein phase (A) does not contain liposoluble vitamins or provitamins corresponding to liposoluble vitamins,
(b) a phase (B) comprising vitamins and/or the corresponding provitamins, wherein the quantity of liposoluble vitamin compounds is larger than the quantity of water soluble vitamin compounds.

Preferably, phase (B) merely contains water soluble vitamins which are not incorporated into phase (A).

In a second embodiment, the present invention is directed to a kit for temporally gradual administration, wherein the kit comprises
(a) a phase (A) comprising water soluble vitamins and/or the corresponding provitamins, wherein phase (A) does not contain liposoluble vitamins or provitamins corresponding to liposoluble vitamins,
(b) a phase (B) comprising vitamins and/or the corresponding provitamins, wherein the quantity of liposoluble vitamin compounds is larger than the quantity of water soluble vitamin compounds.

Preferably, phase (B) merely contains water soluble vitamins which are not incorporated into phase (A).

In the following, preferred features of the embodiments identified above are described in detail.

Preferably, both phases (A) and (B) are formed in a different manner, i.e. comprising a different shape and coloring. It is particularly preferred that both phases are characterized by different colors. Thus, it is preferred in particular that phase (A) is blue and/or phase (B) is red or orange.

The advantage of different shape and/or coloring is that the consumer is able to distinguish both phases from each other.

Phases (A) and (B) particularly represent capsules. Mainly gelatin capsules are preferred. It would be possible that phases (A) and (B) represent tablets, coated tablets, granules, effervescent tablets, powders, dilutions, or further common dosage forms.

It has been found that such two-phase preparations enable the improved absorption of vitamins by the organism because interference (e.g. complexation) of certain vitamin types can be eliminated, especially concerning liposoluble and water soluble vitamins as well as minerals and trace elements.

It is important for the present invention that both phases (A) and (B) are administered temporally gradually, i.e. they are not administered at the same time. In particular, it has been found to be exceedingly beneficial to administer both phases (A) and (B) at intervals of 24 hours.

Therefore, it is particularly advantageous that
(a) phase (A) is taken during meals on days X+2n and
(b) phase (B) is taken during meals on days X+(2n+1).
   where “n” represents nonnegative integral numbers, i.e. 0, 1, 2, 3, 4 etc.

“X” signifies an arbitrary day. In particular, “X” represents the day when administration of the preparation or the kit is started.

In a particular embodiment, phases (A) and (B) are taken at breakfast or dinner, namely on alternating days.

In the following, the individual phases are expanded on.

It is important that both phases (A) and (B) consist of different vitamins to ensure that reciprocal interference of the components is avoided.

Thus, phase (A) comprises water soluble vitamins and/or the corresponding provitamins, wherein phase (A) does not contain liposoluble vitamins or provitamins corresponding to liposoluble vitamins.

The term “provitamins” refers to precursors of vitamins that have to be transformed into the corresponding vitamins to be beneficial for the organism. Thus, e.g. the provitamin of vitamin D (7-dehydrocholesterol) is transformed into vitamin D (cholecalciferol) in the dermis by UV radiation, e.g. by daylight. β-carotin represents the provitamin of retinol (vitamin A), as well.

Vitamins refer to all organic compounds that the human organism does not require for a source of energy, but for functions of vital importance that, for the most part, cannot be synthesized by the human metabolism. Thus, vitamins have to be absorbed with nutrition.

Water soluble vitamins include thiamine (vitamin B₁), riboflavin (vitamin B₂), niacinamid (vitamin B₃), pantothenic acid (vitamin B₅), pyridoxine (vitamin B₆), biotin (vitamin H), folic acid (vitamin B₉), and cobalamin (vitamin B₁₂). Thus, it is particularly desirable that phase (A) contains at least three of these vitamins, but preferentially five, and more preferably all of the water soluble vitamins as mentioned above.

It is desirable concerning phase (B) that this phase mainly contains liposoluble vitamins and/or corresponding provitamins. Though, it would be possible that phase (B) also comprises other vitamins such as water soluble vitamins and/or their corresponding provitamins. The quantity of liposoluble vitamin compounds and/or their corresponding provitamins has to be larger than the quantity of water soluble vitamin compounds and/or their corresponding provitamins in phase (B). In a special embodiment, phase (B) particularly comprises vitamins with an antioxidant impact. According to this, it is preferred that phase (B) at least comprises the liposoluble vitamins retinol (vitamin A) and/or its provitamin betacarotin, tocopherol (vitamin E), and calciferol (vitamin D) as well as the water soluble vitamin ascorbic acid (vitamin C). Preferably, phase (B) comprises exclusively retinol (vitamin A) and/or its provitamin betacarotin, tocopherol (vitamin E), calciferol (vitamin D) and ascorbic acid (vitamin C) as vitamins.

Further liposoluble vitamins may certainly be incorporated into phase (B). Therefore, it is particularly desirable that at least three liposoluble vitamins selected from the group consisting of retinol (vitamin A), calciferol (vitamin D), tocopherol (vitamin E), phylloquinone (vitamin K₁) menaquinone (vitamin K₂), and their provitamins are contained in phase (B). Particularly preferred as liposoluble vitamins are the initially-mentioned tocopherol (vitamin E), retinol (vitamin A) and its provitamin betacarotin, and cholecalciferol (vitamin D).

Moreover, it is conceivable that phase (B) contains two water soluble vitamins, wherein at least one of these is preferably ascorbic acid (vitamin C). It is particularly preferred that phase (B) only contains ascorbic acid (vitamin C) as a water soluble vitamin.

It is also possible that phase (A) and (B), in addition to the vitamins, contain further minerals and trace elements. Thus, a two-phase preparation or a kit of the present invention additionally contain magnesium or zinc and/or green tea in phase (A).

Phase (B) of the two-phase preparation or the kit may preferably additionally comprise zinc gluconate and/or grape rind extract.

It has been found in particular that the separation of zinc and magnesium has a positive impact on the effect on the human organism.

Several factors influence the amount of single vitamins, i.e. if the person is suffering from enhanced stress or possible illnesses. The adequate amount can be determined as necessary. However, it has been found that it is particularly preferred that phase (B) comprises 10-45 mg betacarotin, 15-90 mg tocopherol (vitamin E), 0.001-0.009 mg cholecalciferol (vitamin D), 150-540 mg ascorbic acid (vitamin C), optionally 50-130 mg zinc gluconate, and optionally 60-120 mg grape rind extract.

It has preferably become apparent concerning phase (A) that it comprises 0.1-0.9 mg biotin (vitamin H), 0.2-1.2 mg folic acid (vitamin B₉), 2.0-12.0 mg thiamine mononitrate (vitamin B₁), 2.0-12.0 mg riboflavin-5-phosphate (vitamin B₂), 5.0-45.0 mg niacinamid (vitamin B₃), 5.0-45.0 mg calcium-D-pantothenate (vitamin B₅), 3.0-18.0 mg pyridoxine hydrochloride (vitamin B₆), 0.001-0.01 mg cyanocobalamin (vitamin B₁₂), optionally 200-700 mg magnesium carbonate or 200-700 mg magnesium citrate, optionally 0.03-0.2 mg sodium selenate or 60-300 mg pure magnesium and optionally 20-240 mg green tea.

The two-phase preparation and the corresponding kit respectively described in this invention have been especially approved as dietary supplements. Thus, the present invention is directed to dietary supplements comprising the two-phase preparation as well as dietary supplements consisting of the described kit. Furthermore, the present invention is directed to the use of a phase (A) and a phase (B) as described in the present invention to manufacture a two-phase preparation or a kit as dietary supplement.

The present invention is specified hereafter by the following examples.

### EXAMPLES

The following two-phase preparation has been especially approved:

Blue Capsule:

| | |
|---|---|
| Vitamin H (biotin) 0.3 mg | 200% |
| Vitamin B₉ (folic acid) 0.4 mg | 200% |
| Vitamin B₁ (thiamine mononitrate) 4.0 mg | 286% |
| Vitamin B₂ (riboflavin-5-phosphate) 4.0 mg | 250% |
| Vitamin B₃ (niacinamid) 15 mg | 83.3% |
| Vitamin B₅ (pantothenic acid as calcium D-pantothenate) 15 mg | 250% |
| Vitamin B₆ (pyridoxine hydrochloride 100%) 6.0 mg | 300% |
| Vitamin B₁₂ (cyanocobalamin) 0.002 mg | 200% |
| Magnesium carbonate 240 mg | 20% |
| (equivalent to 60.0 mg magnesium) | |
| Sodium selenate 0.2 mg | *** |
| (equivalent to 0.084 mg selenate) | |
| Green tea 80 mg (95%) | *** |

Orange Capsule:

| | |
|---|---|
| Betacarotin 15 mg | 150% |
| (equivalent to 1200 mcg vitamin A) | |
| Vitamin C (ascorbic acid 97%) 180 mg | 300% |
| Vitamin E (tocopherol acetate) 30 mg | 300% |
| Vitamin D₃ (cholecalciferol) 0.005 mg | 100% |
| Zinc gluconate 66.42-104 mg | |
| (equivalent to 9-12 mg zinc) | |
| Grape rind extract 80 mg | *** |

*** No RDA (recommended daily allowance) values declared

1-23. (canceled)

24. A method of nutritional supplementation comprising the steps of, for at least one, two-day period,
administering to a subject nutritional supplement phase (A) and
administering to the subject nutritional supplement phase (B) on the day after administering nutritional supplement phase (A),
wherein nutritional supplement phase (A) includes water-soluble vitamins, provitamins, or combination thereof, and wherein nutritional supplement phase (A) excludes liposoluble vitamins, provitamins, or combination thereof,
wherein nutritional supplement phase (B) includes a quantity of water-soluble vitamins, provitamins, or combination thereof and a larger quantity of liposoluble vitamins, provitamins, or combination thereof, and
wherein nutritional supplement phase (A) and nutritional supplement phase (B) have mutually distinguishable forms.

25. The method of claim 24, wherein nutritional supplement phase (B) is administered 24 hours after nutritional supplement phase (A).

26. The method according to claim 24, wherein administering is during meals.

27. The method according to claim 24, wherein nutritional supplement phase (B) includes one or two water-soluble vitamins, provitamins, or combination thereof.

28. The method according to claim 24, wherein nutritional supplement phase (B) additionally includes zinc gluconate, grape rind extract, or combination thereof.

29. The method according to claim 24, wherein nutritional supplement phase (A) additionally includes magnesium salts or zinc salts or green tea.

30. The method according to claim 24, wherein nutritional supplement phase (B) includes at least three liposoluble vitamins selected from the group consisting of retinol (vitamin A), cholecalciferol (vitamin D), tocopherol (vitamin E), phylloquinone (vitamin K₁), menaquinone (vitamin K₂), and provitamins thereof.

31. The method according to claim 24, wherein nutritional supplement phase (B) includes ascorbic acid (vitamin C) as the sole water soluble vitamin.

32. The method according to claim 24, wherein nutritional supplement phase (A) includes at least three water soluble vitamins selected from the group consisting of ascorbic acid (vitamin C), thiamine (vitamin B₁), riboflavin (vitamin B₂), niacinamid (vitamin B₃), pantothenic acid (vitamin B₅) pyridoxine (vitamin B₆), biotin (vitamin H), folic acid (vitamin B₉) cobalamin (vitamin B₁₂), and provitamins thereof.

33. The method according to claim 24, wherein the mutually distinguishable forms are different colors.

34. The method according to claim 24, wherein nutritional supplement phase (B) is orange or red.

35. The method according to claim 24, wherein nutritional supplement phase (A) is blue.

36. The method according to claim 24, wherein the mutually distinguishable forms are differently shaped capsules.

37. The method according to claim 24, wherein nutritional supplement phase (B) exclusively includes
a) cholecalciferol (vitamin D) and tocopherol (vitamin E), and either (i) retinol (vitamin A), (ii) provitamin β-carotin, or (iii) both retinol and provitamin β-carotin, as the liposoluble vitamins, provitamins, or combination thereof, and
b) ascorbic acid (vitamin C), as the water-soluble vitamins, provitamins, or combination thereof.

38. The method according to claim 24, wherein nutritional supplement phase (A) exclusively contains thiamine (vitamin B₁), riboflavin (vitamin B₂), niacinamid (vitamin B₃), pantothenic acid (vitamin B₅), pyridoxine (vitamin B₆), biotin (vitamin H), folic acid (vitamin B₉), and cobalamin (vitamin B₁₂) as the water-soluble vitamins, provitamins, or combination thereof.

39. The method according to claim 24, wherein
a) nutritional supplement phase (B) is an orange or red capsule including
i) 10-15 mg β-carotin,
ii) 15-35 mg tocopherol (vitamin E),
iii) 0.001-0.009 mg calciferol (vitamin D),
iv) 150-200 mg ascorbic acid (vitamin C),
b) nutritional supplement phase (A) is a blue capsule including
i) 0.1-0.9 mg biotin (vitamin H),
ii) 0.2-1.2 mg folic acid (vitamin B₉),
iii) 2.0-12.0 mg riboflavin-5-phosphate (vitamin B₂),
iv) 5.0-45.0 mg niacinamid (vitamin B₃),
v) 5.0-45.0 mg calcium-D-pantothenate (vitamin B₅),
vi) 3.0-18.0 mg pyridoxine hydrochloride (vitamin B₆),
vii) 0.001-0.03 mg cyanocobalamin (vitamin B₁₂).

40. The method according to claim 39, wherein
a) nutritional supplement phase (B) further includes
v) a zinc salt is an amount equivalent to about 6-33 mg zinc, and
vi) 60-120 mg grape rind extract and
b) nutritional supplement phase (A) further includes
viii) a magnesium salt in an amount equivalent to 60-300 mg magnesium,
ix) a selenium salt in an amount equivalent to 0.02-0.25 mg selenium, and
x) 20-240 mg green tea.

41. The method of claim 40, wherein the magnesium salt is magnesium citrate or magnesium carbonate, wherein the selenium salt is sodium silenium, and wherein the zinc salt is zinc gluconate.

42. The method according to claim 24, wherein the two-phase preparation is a dietary supplement.

43. A dietary supplement, useful as the nutritional supplement in the method of claim 24, comprising, in separate, mutually distinguishable forms,
a) nutritional supplement phase (A), including water-soluble vitamins, provitamins, or combination thereof, and excluding liposoluble vitamins and provitamins, and
b) nutritional supplement phase (B), including a quantity of water-soluble vitamins, provitamins, or combination thereof and a larger quantity of liposoluble vitamins, provitamins, or combination thereof.

44. The dietary supplement of claim 43, wherein the mutually distinguishable forms are selected from the group consisting of differently colored capsules and tablets, differently shaped capsules and tablets, and differently colored and shaped capsules and tablets.

45. The dietary supplement of claim 43, wherein the mutually distinguishable forms are selected from the group consisting of different colors, different shapes, and different colors and shapes.

46. A kit comprising at least one packaged, nutritional supplement, wherein the nutritional supplement is useful in the method of claim 24 and comprises, in separate, mutually distinguishable forms,
a) nutritional supplement phase (A), including water-soluble vitamins, provitamins, or combination thereof, and excluding liposoluble vitamins, provitamins, or combination thereof, and
b) nutritional supplement phase (B), including a quantity of water-soluble vitamins, provitamins, or combination thereof and a larger quantity of liposoluble vitamins, provitamins, or combination thereof.

47. The kit of claim 46, wherein the mutually distinguishable forms are selected from the group consisting of differently colored capsules and tablets, differently shaped capsules and tablets, and differently colored and shaped capsules and tablets.

48. The kit of claim 46, wherein the mutually distinguishable forms are selected from the group consisting of different colors, different shapes, and different colors and shapes.

49. The kit of claim 47 comprising multiples of the packaged, nutritional supplement.
